(19) United States
(12) Patent Application Publication
(10) Pub. No.: US 2003/0159231 A1
(43) Pub. Date: Aug. 28, 2003

(54) Upright type vacuum cleaner

(76) Inventor: Jang-Keun Oh, Gwangju-City (KR)

Correspondence Address: LADAS & PARRY, 224 SOUTH MICHIGAN AVENUE, SUITE 1200, CHICAGO, IL, 60604, US

(21) Appl. No.: 10/368,752

(22) Filed: Feb. 18, 2003

(30) Foreign Application Priority Data
Feb. 28, 2002 (KR) 2002-10797
Nov. 11, 2002 (KR) 2002-69564

(51) Int. Cl. A47L 7/00

(52) U.S. Cl. 15/320

(57) ABSTRACT

An upright type vacuum cleaner uses an electrolyzed solution instead of detergent, and includes a cleaner body comprising a driving portion for generating a suction force, and a contaminant collecting portion for collecting contaminants drawn in by the suction force of the driving force, a suction brush movably formed on a lower portion of the cleaner body to be moved along a surface to be cleaned for drawing in the contaminants on the surface to be cleaned by the suction force from the driving portion, and a multi-layered electrolyzing device for carrying out an electrolysis by a plurality of electrolytic cells, and for jetting an electrolyzed solution through the suction brush against the surface to be cleaned, the electrolyzed solution being used for removing contaminants on the surface to be cleaned. The multi-layered electrolyzing device includes an electrolyzing portion disposed inside of the cleaner body for generating the electrolyzed solution with a solution as supplied, an electrolyzed solution jetting portion for jetting the electrolyzed solution generated at the electrolyzing portion through the suction brush and against the surface to be cleaned, and a waste water collecting portion for recovering used electrolyzed solution and the contaminants therein after the electrolyzed solution is jetted against the surface to be cleaned and has removed the contaminants on the surface. The electrolyzing portion includes a water tank disposed on an upper side of the cleaner body, first and second electrolyzing tanks connected in series, so that the solution from the water tank is electrolyzed at least two times, and an electrolyzed solution storage tank for temporarily storing the electrolyzed solution which is electrolyzed and fed from the first and the second electrolyzing tanks.

### BACKGROUND OF THE INVENTION

1. Field of the Invention

The present invention generally relates to a vacuum cleaner that cleans a surface to be cleaned by jetting a cleaning solution, and more particularly, it relates to an upright type vacuum cleaner which uses, instead of a detergent, a multi-layered electrolytic component that performs electrolysis with respect to an electrolyte and jets the electrolyzed solution to the surface to be cleaned for cleaning.

2. Description of the Background Art

As is generally known, an upright type vacuum cleaner is constructed in a manner such that a cleaner body having a driving portion therein for generating a suction force is movably mounted on a suction brush that moves along the surface to be cleaned. The brush is rotated on the surface at a predetermined angle.

The cleaner body usually includes filtering means and a contaminant collecting portion. Accordingly, contaminants are drawn in by the suction force of the driving portion, filtered out through the filtering means, and collected in the contaminant collecting portion. A grip is also provided on the upper portion of the cleaner body, having an on/off switch formed thereon. Accordingly, an operator of the cleaner can hold the grip in order to carry out the cleaning operation.

The vacuum cleaner as described above uses suction force to remove dirt and dust from the surface to be cleaned, such as a floor. Such a vacuum cleaner is quite efficient in removing simple dirt or dust. However, it is somewhat inefficient when cleaning stubborn stains on the floor or contaminants disposed on carpets, upholstery, etc. Another conventional vacuum cleaner-performs the cleaning operation with a sweeping action on the floor or carpet through a rotary brush mounted on the suction brush. However, this conventional vacuum cleaner does not completely remove tough stains.

Another conventional vacuum cleaner has a storage tank in the cleaner body for storing a detergent solution therein. When in operation, the detergent solution is fed from the storage tank and then jetted against the floor or carpet, effectively removing stubborn stains and contaminants on the floor or the carpet.

This type of vacuum cleaner typically uses-detergents, which are dissolved in water at a predetermined concentration. Accordingly, it is somewhat troublesome to use such a conventional vacuum cleaner, and maintenance costs are high.

Another problem arises when the detergent feeding path is blocked by the waste byproducts of the detergent, as this causes the cleaner to malfunction.

### SUMMARY OF THE INVENTION

Accordingly, it is an object of the present invention to provide an upright type vacuum cleaner that uses an electrolyte as a cleaning solution which is convenient to use and has reduced maintenance costs, and that also avoids blockage in the path of the cleaning solution.

The above objects are accomplished by providing an upright type vacuum cleaner according to the present invention, including a cleaner body comprising a driving portion for generating a suction force, and a contaminant collecting portion for collecting contaminants drawn in by the suction force of the driving force; a suction brush movably formed on a lower portion of the cleaner body to be moved along a surface to be cleaned for drawing in the contaminants on the surface to be cleaned by the suction force from the driving portion; and a multi-layered electrolyzing device for carrying out an electrolysis by a plurality of electrolytic cells, and for jetting an electrolyzed solution through the suction brush against the surface to be cleaned. The electrolyzed solution is utilized for removing contaminants on the surface to be cleaned.

The multi-layered electrolyzing device includes an electrolyzing portion disposed inside of the cleaner body for generating the electrolyzed solution from a solution supplied from a reservoir disposed in the cleaner body; an electrolyzed solution jetting portion for jetting the electrolyzed solution generated at the electrolyzing portion through the suction brush and against the surface to be cleaned; and a waste water collecting portion for recovering used electrolyzed solution and the contaminants therein after the electrolyzed solution is jetted against the surface to be cleaned and has removed the contaminants on the surface.

The electrolyzing portion includes a water tank disposed on an upper side of the cleaner body; first and second electrolyzing tanks connected in series, so that the solution from the water tank is electrolyzed at least two times; and an electrolyzed solution storage tank for temporarily storing the electrolyzed solution which is electrolyzed and fed from the first and the second electrolyzing tanks.

The electrolyzing portion includes a water tank disposed on an upper side of the cleaner body; an additive tank disposed downstream of the water tank; a mixing tank for mixing and diffusing the additives from the additive tank in the solution from the water tank; a first electrolyzed solution tank and a second electrolyzed solution tank connected in series so that the mixture of the solution and the additive from the mixing tank can be electrolyzed at least two times; and an electrolyzed solution storage tank for temporarily storing the electrolyzed solution discharged from the first and second electrolyzing tanks.

Each of the first and the second electrolyzing tanks is constructed in a layered structure, each comprising at least an anode plate and a cathode plate, and a partition disposed between each anode plate and cathode plate.

There is a reservoir disposed between the first and the second electrolyzing tanks.

The reservoir separately stores an anolyte and a catholyte discharged from the first electrolyzing tank, and the anolyte is fed into an anode cell of the second electrolyzing tank, while the catholyte is fed into a cathode cell of the second electrolyzing tank. Alternatively, the anolyte can be fed into a cathode cell of the second electrolyzing tank, while the catholyte is fed into an anode cell of the second electrolyzing tank.

A gas removing device may be attached to the upper portion of the reservoir, for removing any gas from the electrolyzed solution that may be generated by the electrolyzing process.

The electrolyzed solution jetting portion includes an electrolyzed solution conveyance path connecting the electrolyzing portion to a leading end of the suction brush; a jetting pump disposed in the electrolyzed solution conveyance path; and a nozzle provided at an end of the electrolyzed solution conveyance path for jetting the electrolyzed solution therethrougb.

The waste water collecting portion is arranged in the discharging path of the cleaner body.

### BRIEF DESCRIPTION OF THE DRAWINGS

The above-mentioned objects and the features of the present invention will be made more apparent by describing the preferred embodiment of the present invention in detail referring to the appended drawings, in which:

FIG. 1 is a front view, in partial cutaway, showing an upright type vacuum cleaner according to a preferred embodiment of the present invention;

FIG. 2 is a block diagram schematically showing one example of the main aspect of the present invention, i.e., a multi-layered electrolytic device and connecting pipes thereof;

FIG. 3 is a block diagram schematically showing the multi-layered electrolytic device according to a preferred embodiment of the present invention; and

FIG. 4 is an exploded perspective view showing the structure of the multi-layered electrolytic device according to the present invention.

### DETAILED DESCRIPTION OF THE PREFERRED EMBODIMENTS

The objects and other characteristics of the present invention will be made more apparent by describing the preferred embodiments with reference to the accompanying drawings.

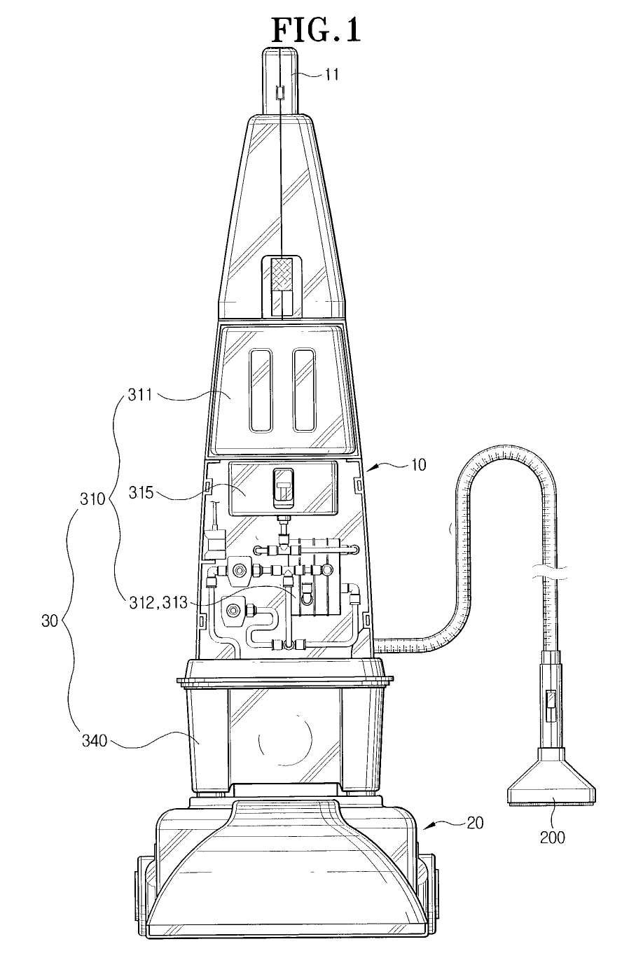

As shown in FIG. 1, an upright type vacuum cleaner according to a preferred embodiment of the present invention is constructed in a manner such that a cleaner body 10 and a suction brush 20 are movably connected with each other to be rotated at a predetermined angle relative to each other, and a multi-layered electrolytic device 30 is formed in the cleaner body 10.

An operator of the vacuum cleaner performs the cleaning job while moving the cleaner by a grip 11 that is formed on the upper portion of the cleaner body 10. Accordingly, dirt or dust are drawn into the cleaner body 10 through the suction brush 20, which moves along the surface to be cleaned, such as a floor. Albeit not shown in detail, in the cleaner body 10, there are a driving portion that provides a suction force and also a contaminant receptacle. Accordingly, contaminants drawn in through the suction brush 20 are collected in the contaminant receptacle, while the clean air is discharged to the outside.

The multi-layered electrolytic device 30 electrolyzes the electrolyte and jets such electrolyzed solution as a cleaning solution for removing the tough stains on the floor or the contaminants on the carpet, upholstery, etc. By the chemical action with the electrolyte, stains or contaminants are effectively removed. According to the present invention, an electrolyte is used as a cleaning solution instead of conventional detergents. Accordingly, many problems that arise while using of detergents can be prevented, and maintenance cost is reduced. Also, no blockage of the path of the cleaning solution occurs.

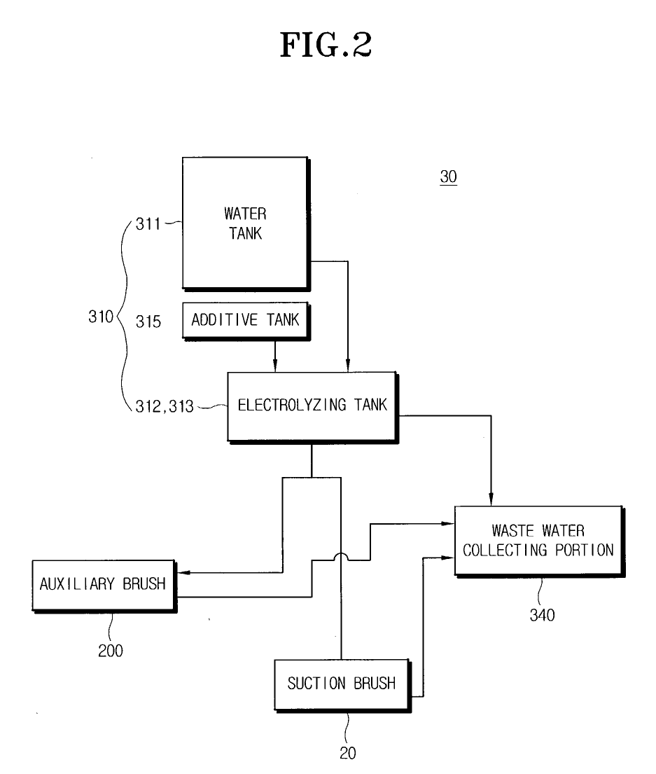

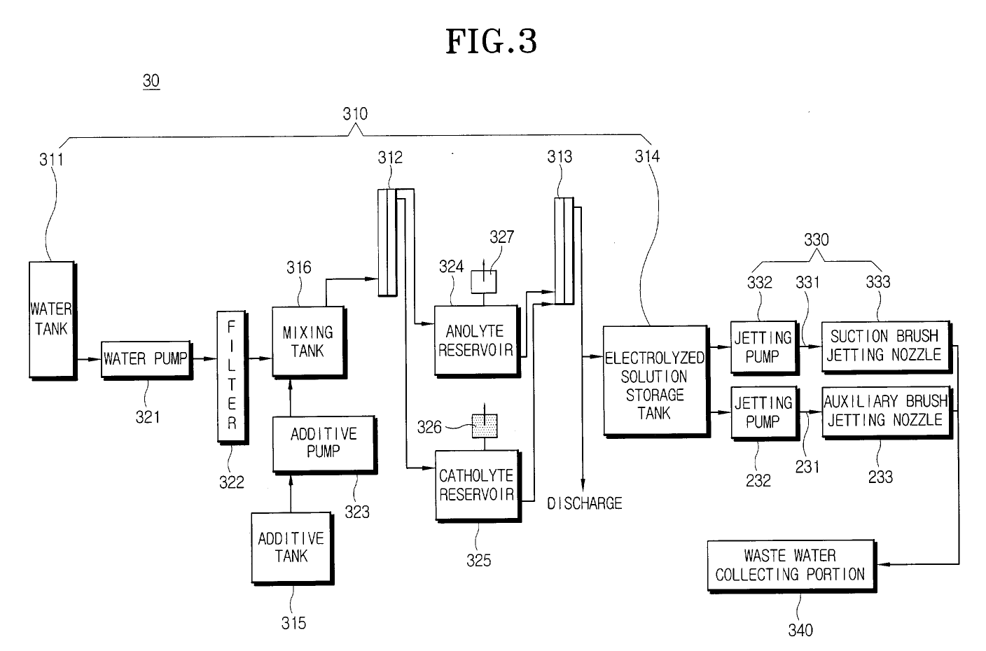

Referring to FIGS. 2 and 3, the multi-layered electrolytic device 30 is mounted inside of the cleaner body 10 (FIG. 1), and includes an electrolyzing portion 310 that carries out electrolysis with the supplied water, an electrolyzed solution jetting portion 330 for jetting the electrolyzed solution from the electrolyzing portion 310 to the surface to be cleaned through the suction brush 20, and a waste water collecting portion 340 for recovering and storing the used electrolyzed solution and the contaminants migrated from the surface to be cleaned therein.

The electrolyzing portion 310 includes a water tank 311 mounted on the upper side of the cleaner body 10, a first electrolyzing tank 312 and a second electrolyzing tank 313 connected in series to subject the water from the water tank 311 to electrolysis in at least two stages, and an electrolyzed solution storage tank 314 for temporarily storing the electrolyzed solution discharged from the first and the second electrolyzing tanks 312, 313.

According to the preferred embodiment of the present invention, the electrolyzing portion 310 further includes an additive tank 315 arranged downstream of the water tank 311, and a mixing tank 316 for mixing and diffusing the additives of the additive tank 315 in the water from the water tank 311.

The water tank 311 stores the water used in the electrolysis process. Such water may be obtained from a variety of sources, such as tap water, fresh water, seawater or groundwater. The water is stored in the water tank 311 and flows from-the water tank 311 into the mixing tank 316 via the filter 322. Preferably, the water tank 311 includes a metering pump 321. The filter 322 filters out ions or impurities in the water.

The additive tank 315 stores additives to be dissolved in the water for electrolysis, such as sodium chloride, potassium chloride, and calcium chloride. The additive tank 315 is connected with the mixing tank 316 by pipes, and includes an additive pump 323 formed in the fluid communication path thereof. It is preferred that the pipe connecting the additive tank 315 and the mixing tank 316 is directly connected to the pipe connecting the filter 322 and the mixing tank 316, or at least connected to the proximity of the pipe connecting the filter 322 and the mixing tank 316. In an alternative construction, the additive tank 315 and the additive pump 323 may be omitted when the electrolysis can be carried out without requiring additives.

The pipe drawn out from the upper portion of the mixing tank 316 is connected to the lower portion of the first electrolyzing tank 312. The electrolysis is carried out in the layered structure, i.e., it is carried out in the anode cell and the cathode cell, which are partitioned off from each other with a layer.

The electrolysis is carried out in the first electrolyzing tank 312 with the water supplied from the mixing tank 316, and the first electrolyzed solution is fed through the upper portion of the first electrolyzing tank 312. The first electrolyzed solution is fed out of the first electrolyzing tank 312 by two pipe lines as the water is fed into the first electrolyzing tank 312. Catholyte is generated in the cathode cell, and anolyte is generated in the anode cell. During the electrolysis in the first electrolyzing tank 312, gas (mostly, hydrogen gas) is generated in the catholyte. Accordingly, it is preferred to feed the first electrolyzed solution through the upper portion of the first electrolyzing tank 6312, thereby to prevent the gas from remaining in the first electrolyzing tank 312.

The first electrolyzed solution, i.e., the anolyte and the catholyte each flow into an anolyte reservoir 324 and a catholyte reservoir 325, respectively, located between the first and the second electrolyzing tanks 312, 313. Being located between the first and the second electrolyzing tanks 312, 313, the anolyte and the catholyte reservoirs 324, 325 also act to regulate the flow rate of the first electrolyzed solution from the first electrolyzing tank 312. Since the gas generated during the electrolysis process remains in the catholyte of the first electrolyzed solution, the gas is collected in the upper portion of the catholyte reservoir 325. A gas removing device 326 may be disposed at the upper portion of the catholyte reservoir 325 to remove the gas. Further, inverse voltage is frequently applied to remove scaling from the anode and cathode of the first electrolyzing tank 312, and sometimes the catholyte is introduced into the anode reservoir 324 to work together with the application of the inverse voltage. In such case, another gas removing device 327 can be disposed at the upper portion of the anolyte reservoir 324. Any generally known device that only permits the gaseous substance to be removed, but not the water, can be used as the gas removing devices 326, 327. Another form of gas removing device can also be used, which measures the amount of gas collected in the reservoirs 324, 325 and then opens a valve to vent the reservoirs as the amount of gas exceeds a predetermined limit.

In FIG. 3, the anolyte reservoir 324 and the catholyte reservoir 325 are divided from each other. However, the anolyte reservoir 324 and the catholyte reservoir 325 can be formed as one body, which is divided into two inner parts by an inner partition.

The first electrolyzed solutions from the reservoirs 324, 325 are fed to the second electrolyzing tank 313 through the lower portion. The anolyte from the anolyte reservoir 324 is fed into the anode cell of the second electrolyzing tank 313, while the catholyte from the catholyte reservoir 325 is fed into the cathode cell of the second electrolyzing tank 313. As in the first electrolyzing tank 312, the first electrolyzed solution is subject to a second electrolysis in the second electrolyzing tank 313. More specifically, the electrolysis is additionally carried out in the second electrolyzing tank 313 with respect to the electrolyte, which is not electrolyzed in the first electrolyzing tank 312. As in the first electrolyzing tank 312, a gas is generated in the cathode cell of the second electrolyzing tank 313 during the electrolysis, and thus, it is preferred that the second electrolyzed solution is also fed out through the upper portion of the second electrolyzing tank 313.

After being fed out from the second electrolyzing tank 313, the second electrolyzed solution flows to, and is stored in, the electrolyzed solution tank 314. The electrolyzed solution tank 314 separately stores the anolyte from the catholyte, and as necessary, the electrolyzed solution tank 314 can store the anolyte separately from the catholyte in the anolyte tank and the catholyte tank, respectively.

A discharge valve is disposed between the second electrolyzing tank 313 and the electrolyzed solution tank 314 for discharging the electrolyzed solution, as shown.

Although FIG. 3 shows the multi-layered electrolyzing device as having two electrolyzing tanks 312, 313 and the anolyte reservoir 324 and the catholyte reservoir 325, the electrolyzing tanks and the reservoirs can be provided with a larger number of tanks and reservoirs connected in series. Further, electrolysis efficiency can be increased by connecting a set of electrolyzing tanks and reservoirs in parallel.

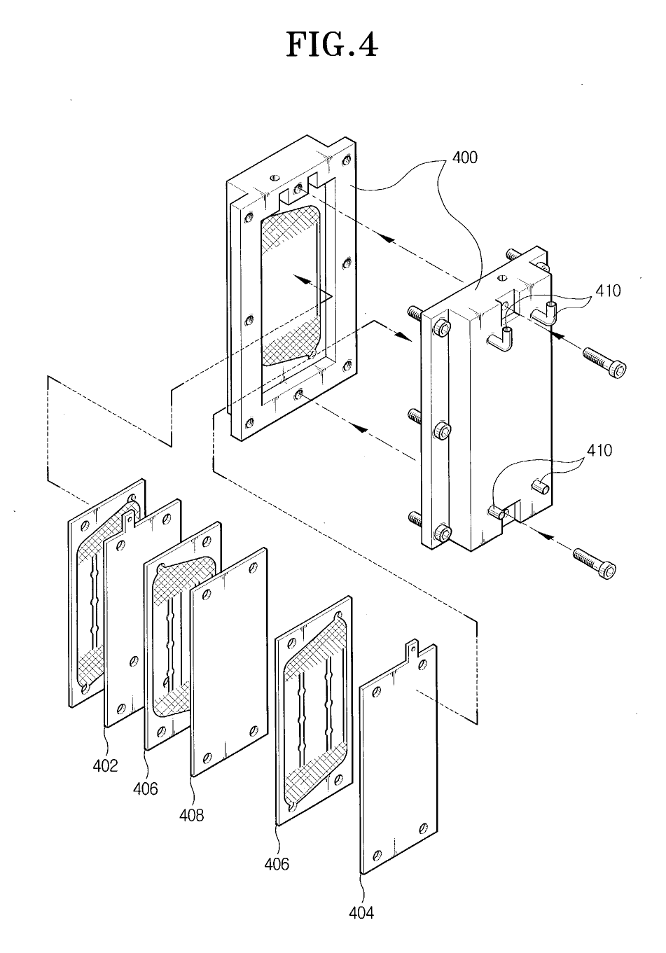

As shown in FIG. 4, each of the first and the second electrolyzing tanks 312, 313 includes a housing 400, an anode plate 402, a cathode plate 404, two spacers 406 sandwiched between the anode plate 402 and the cathode plate 404, a partition 408 sandwiched between the two spacers 406, and pipes 410 for feeding in, or discharging out, the water with respect to the first and the second electrolyzing tanks 312, 313.

The housing 400 secures the anode plate 402, the cathode plate 404, the spacers 406, the partition 408 and the pipe 410 therein.

The anode plate 402 and the cathode plate 404 are made of a titanium plate on which iridium is coated. Alternatively, a titanium plate coated with a platinum coating can also be used for the plates 402, 404. Of course, a standard metal plate or a carbon plate can be used, or a steel plate may be recommended for use, rather than the titanium plate coated with iridium, if manufacturing cost is considered a factor.

The spacers 406 function to insulate the anode plate 402 and the cathode plate 404, and to define a path for the supplied water. The spacers 406 are designed such that the water path remains in contact with the anode plate 402 and the with cathode plate 404, which are attached as close as possible to the sides of the spacers 406. The spacers 406 can have various shapes.

Due to the presence of the partition 408, ion exchange occurs in the water disposed in the spaces defined by the spacers 406, without direct mixture of the water. The number of anode plates, cathode plates and intervening spacers need not be unlimited, as is necessary.

Referring now to FIG. 3, the electrolyzed solution jetting portion 330 includes an electrolyzed solution conveyance path 331 connected to the electrolyzing portion 310, and more specifically, from the electrolyzed solution storage tank 314 to a leading end of the suction brush 20 providing fluid communication therebetween. A jetting pump 332 may be disposed in the electrolyzed solution conveyance path 331, and a jetting nozzle 333 may be formed at an end of the electrolyzed solution conveyance path 331.

According to the preferred embodiment of the present invention, the vacuum cleaner includes not only the suction brush 20, but also an auxiliary brush 200. The auxiliary brush 200 is connected to the driving portion of the cleaner body 10, and the electrolyzed solution from the electrolyzing portion 310 is fed through the auxiliary brush 200 to the surface to be cleaned. As shown in FIG. 3, a separate electrolyzed solution conveyance path for feeding the electrolyzed solution through the auxiliary brush 200 includes a jetting pump 232 and a jetting nozzle 233. Albeit not shown in detail, a recovery path is also formed in the hose connecting the auxiliary brush 200 to the cleaner body 10, so as to convey the used electrolyzed solution and the contaminants therein to the waste water collecting portion 340.

The waste water collecting portion 340 includes a contaminant receptacle which is arranged on the discharge path of the cleaner body 10. Accordingly, the used electrolyzed solution and the contaminants therein are drawn into the cleaner body 10 through the suction brush 20, and the used electrolyzed solution and the wet contaminants are collected in the waste water collecting portion 340 while the rest of contaminants are collected in the contaminant receptacle of the cleaner body 10. The clean air is discharged to the outside.

With an upright type vacuum cleaner having a multi-layered electrolytic device constructed as described above according to the present invention, an electrolyzed solution from the electrolyzing portion is jetted against the floor or carpet to be cleaned. By chemical action with the electrolyzed solution as it is being jetted, the tough stains or contaminants disposed on the floor or carpet are effectively removed. As used here, the “electrolyzed solution” is one that is generally used for treating waste water, bleaching cotton fabrics, and disinfecting and sterilizing a water supply. Without requiring the addition of detergent, the electrolyzed solution can effectively remove the stubborn stains and contaminants disposed on the floor or the carpet.

Accordingly, the operator does not experience the inconvenience of having to dissolve the detergent in the water, and he/she also does not need to buy the detergent, thus lowering operating costs. As described above, by having the electrolyzing device, the cleaning of old stains and contaminants in the floors and the carpets can be effectively carried out without having to use detergents. Also, all the ions present in the water can be electrolyzed by the multi-layered electrolyzing device according to the present invention. Accordingly, an electrolyzed solution having excellent cleaning properties is generated, and the maintenance cost of the vacuum cleaner decreases. The vacuum cleaner of the present invention is easier to use. Further, since the vacuum cleaner having the electrolyzing device according to the present invention does not use a detergent, the blockage of the cleaning solution path with detergent can be avoided.

Although preferred embodiments of the present invention have been described, it will be understood by those skilled in the art that the present invention should not be limited to the described preferred embodiments, but various changes and modifications can be made while remaining within the spirit and scope of the present invention as defined by the appended claims and their equivalents.

What is claimed is:

1. An upright type vacuum cleaner, comprising:
a cleaner body comprising a driving portion for generating a suction force, and a contaminant collecting portion for collecting contaminants drawn in by the suction force of the driving force;
a suction brush movably formed on a lower portion of the cleaner body to be moved along a surface to be cleaned for drawing in the contaminants on the surface to be cleaned by the suction force from the driving portion; and
a multi-layered electrolyzing device for carrying out an electrolysis by a plurality of electrolytic cells, and for jetting an electrolyzed solution being used through the suction brush against the surface to be cleaned, the electrolyzed solution being used for removing contaminants on the surface to be cleaned.

2. The upright type vacuum cleaner of claim 1, wherein the multi-layered electrolyzing device comprises:
an electrolyzing portion disposed inside of the cleaner body, for generating the electrolyzed solution from a solution supplied from a reservoir disposed in the cleaner body;
an electrolyzed solution jetting portion for jetting the electrolyzed solution generated at the electrolyzing portion through the suction brush and against the surface to be cleaned; and
a waste water collecting portion for recovering used electrolyzed solution and the contaminants therein after the electrolyzed solution is jetted against the surface to be cleaned and has removed the contaminants on the surface.

3. The upright type vacuum cleaner of claim 2, wherein the electrolyzing portion comprises:
a water tank disposed on an upper side of the cleaner body;
first and second electrolyzing tanks connected in series, so that the solution from the water tank is electrolyzed at least two times; and
an electrolyzed solution storage tank for temporarily storing the electrolyzed solution which is electrolyzed and fed from the first and the second electrolyzing tanks.

4. The upright type vacuum cleaner of claim 2, wherein the electrolyzing portion comprises:
a water tank disposed on an upper side of the cleaner body;
an additive tank disposed downstream of the water tank;
a mixing tank for mixing and diffusing the additives from the additive tank in the solution from the water tank;
a first electrolyzed solution tank and a second electrolyzed solution tank connected in series so that the mixture of the solution and the additive from the mixing tank can be electrolyzed at least two times; and
an electrolyzed solution storage tank for temporarily storing the electrolyzed solution discharged from the first and second electrolyzing tanks.

5. The upright type vacuum cleaner of claim 3, wherein each of the first and the second electrolyzing tanks is each constructed in a layered structure comprising at least an anode plate and a cathode plate, and a partition disposed between the anode plate and the cathode plate.

6. The upright type vacuum cleaner of claim 5, wherein there is a reservoir disposed between the first and the second electrolyzing tanks.

7. The upright type vacuum cleaner of claim 6, wherein the reservoir separately stores an anolyte and a catholyte discharged from the first layered electrolyzing tank, and
the anolyte is fed into an anode cell of the second electrolyzing tank, while the catholyte is fed into a cathode cell of the second electrolyzing tank.

8. The upright type vacuum cleaner of claim 6, wherein the reservoir separately stores an anolyte and a catholyte discharged from the first electrolyzing tank, and
the anolyte is fed into a cathode cell of the second electrolyzing tank, while the catholyte is fed into an anode cell of the second electrolyzing tank.

9. The upright type vacuum cleaner of claim 6, wherein a gas removing device is attached to the upper portion of the reservoir, for removing gas generated by the electrolysis process from the electrolyzed solution.

10. The upright type vacuum cleaner of claim 2, wherein the electrolyzed solution jetting portion comprises:
an electrolyzed solution conveyance path connecting the electrolyzing portion to a leading end of the suction brush;
a jetting pump disposed in the electrolyzed solution conveyance path; and
a nozzle provided at an end of the electrolyzed solution conveyance path for jetting the electrolyzed solution therethrough.

11. The upright type vacuum cleaner of claim 2, wherein the waste water collecting portion is arranged in the discharging path of the cleaner body.
